(19) United States
(12) Patent Application Publication
(10) Pub. No.: US 2023/0157859 A1
(43) Pub. Date: May 25, 2023

(54) FECAL DIVERSION TUBE SYSTEM

(71) Applicant: Tarek Hassab, Ellicott City, MD (US)

(72) Inventor: Tarek Hassab, Ellicott City, MD (US)

(21) Appl. No.: 17/993,107

(22) Filed: Nov. 23, 2022

Related U.S. Application Data
(60) Provisional application No. 63/282,309, filed on Nov. 23, 2021.

(51) Int. Cl. A61F 5/44; A61M 1/00; A61F 5/445

(52) U.S. Cl. CPC A61F 5/4407; A61M 1/84; A61F 5/445; A61M 2210/106; A61M 2210/1064

(57) ABSTRACT

A fecal diversion tube system. Such a system includes an inflatable balloon, a fluid tube, and a fenestrated tube that bifurcates into a fluid port and an egress port. The system may be used to protect or ameliorate an anastomotic leak and may replace or reduce the need for diverting loop ileostomy following rectal resection.

### CROSS REFERENCE TO RELATED APPLICATIONS

This application claims the benefit of U.S. Provisional Application Ser. No. 63/282,309, filed Nov. 23, 2021, the disclosure of which is incorporated herein by reference in its entirety.

### BACKGROUND OF THE INVENTION

As part of the treatment for colorectal cancer or perforated diverticulitis, patients may require a surgical removal of the rectum or rectosigmoid junction and connection (also known as anastomosis) of the colon and rectum together. This is known as colorectal anastomosis. The surgery may be followed by a diverting loop ileostomy (DLI) in cases where the anastomosis is at a high risk for leak. For example, in patients receiving radiotherapy for cancer and patients with feculent peritonitis after perforated diverticulitis, DLI is performed to protect the new connection from the morbidity of anastomotic leaks should they happen. A DLI diverts enteric content through the anterior abdominal wall giving the new connection time to heal. After providing appropriate time for the connection to heal, the anastomosis is evaluated and if it is well healed with no leak or stenosis, the ileostomy may be surgically reversed. Therefore, ileostomies are not benign; there are a range of complications including dehydration, acute renal failure, skin excoriation, and hernia. Some studies show that DLI on its own does not reduce the incidence of leak but rather the morbidity associated with leakage. Others show that DLI can reduce the incidence of anastomotic leak and resulting need for surgery if a leak does occur. The exact reason why some anastomoses leak while others do not is unknown. Contributing factors include blood supply, tension, radiation, sepsis, blood pressure, microbiome, smoking, obesity, alcohol, pre-operation steroids, non-steroidal anti-inflammatory drugs (NSAIDs), blood transfusions, operative duration, case volume, and other factors.

Current alternatives to DLI do not have enough evidence to replace DLI as a treatment for anastomotic leaks. Alternatives include decompression devices, intracolonic devices, and biodegradable devices. A decompression device functions by keeping the anal sphincter open which lowers intraluminal pressure and pressure on the anastomosis. Intracolonic devices prevent fecal load from contacting the anastomotic site, preventing leakage into the peritoneal cavity. Biodegradable devices are composed of materials which after protecting the anastomosis from enteric content, spontaneously dissolve and therefore do not require surgical intervention. These devices are utilized proximal to the anastomosis, but do not occlude the ileocecal junction. In addition, research on the effectiveness of these alternatives is lacking. Issues include challenging and invasive insertion, risk of disruption to the anastomosis, risk of dislocation, and lack of practicality for patients. Consequently, experts have not found or agreed upon an appropriate alternative and so DLI remains the standard of care to protect high risk colorectal anastomoses.

### DESCRIPTION OF THE FIGURES

FIG. 1 illustrates an embodiment of the disclosed fecal diversion tube system within a cross section of the ileum and cecum, with the non-fenestrated segment extending through an extraction site with the fluid port and gastrointestinal content egress port on the exterior of a patient.

FIG. 2 illustrates a three-dimensional rendering of an embodiment of the disclosed fecal diversion tube system with the balloon, fenestrated segment with ileal openings, non-fenestrated segment, and egress drainage port and fluid port.

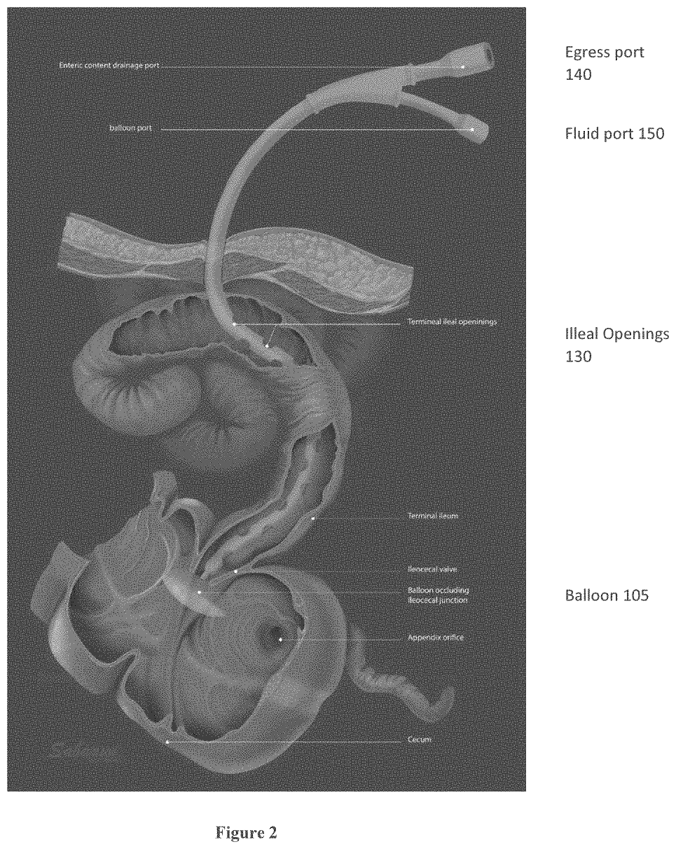

### DETAILED DESCRIPTION OF THE INVENTION

A preferred embodiment of the present invention is now described with reference to the figures, where like reference numbers indicate identical or functionally similar elements. Also in the figures, the leftmost digit of each reference number corresponds to the figure in which the reference number is first used. While specific configurations and arrangements are discussed, it should be understood that this is done for illustrative purposes only. A person skilled in the relevant art will recognize that other configurations and arrangements can be used without departing from the spirit and scope of the invention. It will be apparent to a person skilled in the relevant art that this invention can also be employed in other systems and applications.

The disclosed invention comprises a fecal diversion tube system to protect or ameliorate an anastomotic leak after a colorectal anastomosis. It utilizes the size discrepancy between the ileum and the colon at the ileocecal junction to divert enteric contents. This system may replace or reduce the need for DLI following rectosigmoid resection.

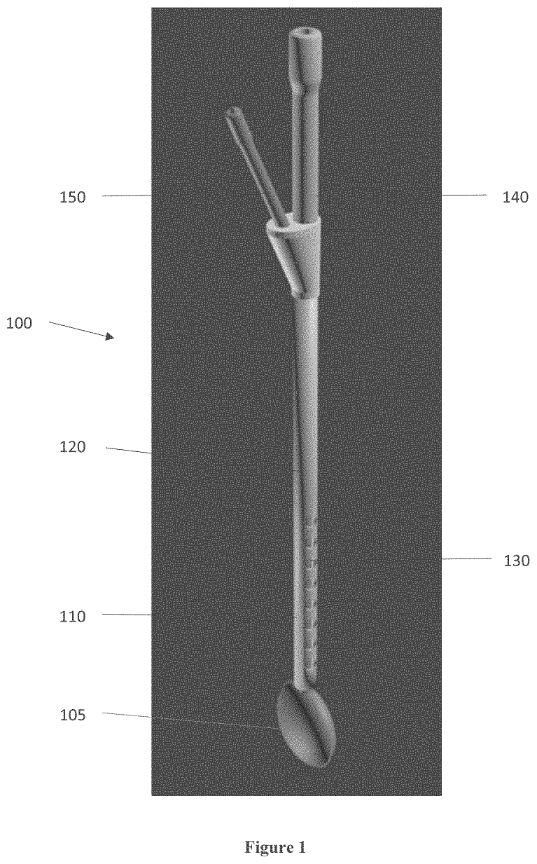

The disclosed invention, shown in FIG. 1 according to an embodiment 100, comprises an inflatable balloon 105 and a fluid tube 110 upstream from the balloon 105. The fluid tube 110 is in fluid communication with balloon 105. At a proximal end of fluid tube 110 is a fluid port 150 which is used to inflate and deflate the balloon 105 via fluid tube 110. A fenestrated tube 120 is positioned generally parallel to fluid tube 110. The fenestrated tube 120 has ileal openings 130 which allow enteric content (gastrointestinal material) to enter the tube for evacuation via a gastrointestinal material egress port 140 when suction is applied.

An embodiment of the disclosed invention comprises of a 20 cm fenestrated segment with ileal openings 0.5 cm in diameter, and a 10 cm non-fenestrated segment. These dimensions may vary in alternative embodiments. Embodiments of the tube system may be constructed of one or more of silicon, rubber, or other related material known or used by a person with skill in the relevant art.

The disclosed invention utilizes the size discrepancy between the small intestine and large intestine. The ileum, part of the small intestine, connects to the cecum, the first part of the large intestine. The ileum has a narrower diameter than the cecum. The fecal diversion tube system is inserted into the ileum with the distal end attached to the inflatable balloon 105 passed through the ileocecal valve and into the cecum. The proximal tube end with egress port 140 and fluid port 150 remains external to the body. The balloon 105 is inflated in the cecum and the device is pulled back to occlude the ileocecal junction. This prevents further passage of intestinal contents.

The apparatus may be implanted in a patent in the manner described here, according to an embodiment. A loop of ileum approximately 20 cm proximal to ileocecal junction is chosen to be the site of diversion. It is confirmed to be sufficiently mobile to reach the chosen site without tension. A stay suture with 3-0 Vicryl is placed to mark the proximal part of the ileum at 25 cm from ileocecal junction and a 3-0 silk is used to mark the distal part. A purse-string suture using 3-0 silk is placed on the antimesenteric border around the proximal stay suture. The fecal diversion tube is then passed through the chosen site on the right lower quadrant of the anterior abdominal wall and the balloon 105 is tested to make sure it is functioning well. An incision is made with electrocautery in the intestinal wall in the center of the purse-string suture. A hemostat clamp is used to expand the enterotomy and confirm entry into the ileal lumen. The fecal diversion tube is then inserted into the lumen of the ileum and advanced distally until the balloon 105 of the tube is all the way through ileocecal valve and inflated with 5 cc of normal saline and confirmed to be within the cecum. The tube 110 with the inflated balloon 105 is then pulled back until it is snug against the ileocecal junction. The purse-string suture is secured in place. The ileum is then tacked to the anterior abdominal wall at the catheter entrance site with 3-0 silk×4 sutures around the tube in such a manner as to prevent torsion or leak. Egress port 140 is then connected to a collection bag that may be hooked to low intermittent wall suction (−80 mmhg) and flushed with normal saline 10 cc 4 times a day) to ensure it is not clogged.

In some embodiments, the balloon 105 may be placed in line, perpendicular or orthogonal to the axis of insertion. Following the recovery period after the colorectal anastomosis, if the connection heals and there is no leak, the balloon 105 may be deflated, and the fecal diversion tube system may be extracted through an extraction site. This may not require surgical intervention. Instead, the tube may be pulled out and the extraction site may be covered in gauze to heal spontaneously. This saves patients from the risks and complications associated with DLI and negates the need for a surgery to close the DLI.

While specific configurations and arrangements have been discussed, it should be understood that this is done for illustrative purposes only. A person skilled in the relevant art will recognize that other configurations and arrangements can be used without departing from the spirit and scope of the invention.

What is claimed is:

1. An apparatus, comprising:
an inflatable balloon;
a fluid tube connected to and in fluid communication with the balloon, wherein the fluid tube is connected to the balloon at a distal end of the fluid tube, and wherein a proximal end of the fluid tube comprises a fluid port configured to permit a fluid to flow into and out of the balloon via the fluid tube; and
a fenestrated tube alongside the fluid tube, and wherein a proximal end of the fenestrated tube comprises an egress port configured to permit egress of gastrointestinal (GI) material via the fenestrated tube.

2. The apparatus of claim 1, wherein the balloon is configured to occlude an ileocecal junction of a patient and wherein the fenestrated tube is upstream from and proximal to the balloon located within the last 15-20 cm of a terminal ileum after which the fenestrated tube exits the ileum through an enterostomy and an anterior abdominal wall to the exterior of the patient.

3. The apparatus of claim 1, wherein the balloon is configured to be inflated and deflated through the fluid port and, when inflated, occludes an ileocecal junction of a patient, and wherein the fluid port is located external to the patient.

4. The apparatus of claim 1, wherein the egress port is configured to receive suction to extract GI material.
